SEQUENCE LISTING (1 sequence)

SEQ ID NO: 1 - DNA, 1098 bp, Homo sapiens
  source 1..1098: mol_type = genomic DNA
atggccgtcatggcgccccgaaccctcctcctgctactctcgggggccctggccctgacccagacctgggcgggctcccactccatgaggtatttcttcacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctactacaaccagagcgaggccggttctcacaccatccagataatgtatggctgcgacgtggggtcggacgggcgcttcctccgcgggtaccggcaggacgcctacgacggcaaggattacatcgccctgaacgaggacctgcgctcttggaccgcggcggacatggcggctcagatcaccaagcgcaagtgggaggcggcccatgaggcggagcagttgagagcctacctggatggcacgtgcgtggagtggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtccctcacagcttgtaaagtgtga

(19) United States
(12) Patent Application Publication
(10) Pub. No.: US 2025/0069688 A1
(43) Pub. Date: Feb. 27, 2025

(54) METHOD OF HUMAN LEUKOCYTE ANTIGEN LOSS OF HETEROZYGOSITY DETECTION IN LIQUID BIOPSIES

(71) Applicant: The USA, as represented by the Secretary, Department of Health and Human Services, Bethesda, MD (US)

(72) Inventors: Andrew SINKOE, Bethesda, MD (US); James GULLEY, Bethesda, MD (US); Christian HINRICHS, Bethesda, MD (US); Scott Alberto NORBERG, Bethesda, MD (US); Clint ALLEN, Bethesda, MD (US); Nisha NAGARSHETH, Bethesda, MD (US); Xiaolin WU, Bethesda, MD (US); Ling SU, Bethesda, MD (US)

(21) Appl. No.: 18/729,138

(22) PCT Filed: Jan. 13, 2023
(86) PCT No.: PCT/US2023/060664

§ 371 Date: Jul. 15, 2024

Related U.S. Application Data
(60) Provisional application No. 63/299,672, filed on Jan. 14, 2022.

(51) Int. Cl. G16B 20/10; G16B 20/20; G16B 30/10; G16H 20/17

(52) U.S. Cl. CPC G16B 20/10; G16B 20/20; G16B 30/10; G16H 20/17

(57) ABSTRACT

Provided herein are methods of determining loss of heterozygosity in human leukocyte antigen in liquid biopsies and applications thereof in cancer treatment.

### STATEMENT REGARDING FEDERALLY SPONSORED RESEARCH OR DEVELOPMENT

This invention was made in part with Government support under NIH grant no. ZIA BC 011871. The Government has certain rights in this invention.

### FIELD OF THE INVENTION

The present invention relates to methods of determining loss of heterozygosity in human leukocyte antigen in liquid biopsies and applications thereof in cancer treatment.

### BACKGROUND OF THE INVENTION

Human Leukocyte Antigen (HLA) genes code for Major Histocompatibility (MHC) proteins, which present antigens on the surface of most cells in the body, including cancer cells. These antigens are presented on the cell surface as part of the immune system, for T cells to recognize and destroy cells containing foreign peptides. There are two groups of HLA genes: HLA class I and HLA class II. The HLA class I group includes the genes HLA-A, HLA-B, HLA-C, and others. The HLA class II group includes the genes HLA-DPB1, HLA-DQB1, HLA-DRB1, and others. Cytotoxic CD8+ T cells are generally known to engage with the MHC proteins coded by HLA class I genes, while helper CD4+ T cells are generally known to engage with the MHC proteins coded by HLA class II genes. All of the HLA genes are located on Chromosome 6 (FIG. 1).

HLA genes are highly polymorphic. That is, many different alleles for each gene are observed in the human population. For example, there are currently 7644 known HLA-A alleles and 9097 HLA-B alleles in the human population, and the number continues to grow as more individuals' HLA genes are sequenced for medical applications including transplant and cancer treatment.

Because of the highly polymorphic nature of the HLA genes, most individuals are heterozygous for their HLA genes, i.e., having two different alleles for each of the HLA genes. For example, a given individual may have one HLA-A allele denoted by HLA-A*02:01:01:01 and the individual's other homologous HLA-A allele might be denoted by HLA-A*03:01:01:01. It is possible for an individual to be homozygous for at least one of their HLA genes, and this occurs in approximately 1/10 of individuals.

Because each T cell in a human's immune system targets a specific antigen bound to a specific MHC protein coded by a specific HLA gene and allele, a given T cell will only be able to destroy an infected or malignant cell if that target cell expresses the T cell's target peptide and MHC on its surface membrane. It has been shown that cancers can delete HLA genes and alleles from their genome that would otherwise allow T cells to recognize and destroy the cancer cells. This has been demonstrated by multiple clinical studies as a resistance mechanism to T-cell based immunotherapy. Any therapy that directly or indirectly involves a T-cell receptor (TCR) of a T cell recognizing a cancer cell via the cancer cell's MHC-peptide complex is susceptible to resistance caused by the cancer deleting the HLA gene and allele that the T cell would normally bind to. When one of a patient's HLA alleles is deleted in their cancer's genome, the phenomenon is called HLA Loss of Heterozygosity (HLA LOH).

HLA LOH can delete multiple HLA genes simultaneously by deleting a segment of Chromosome 6 that covers more than one of the HLA genes. A multiple deletion of the major class I genes (HLA-A, HLA-B, and HLA-C) is somewhat common because these three genes are adjacent to each other on the chromosome; it was found in a large-scale study that in 85% of patients where HLA LOH was detected, the deletion included all three of the major HLA class I genes. It is also possible for cancer genomes to have deletions of only one or two of the major HLA class I genes. Because the HLA class II genes are also located adjacent to the HLA class I genes, it is possible for a cancer genome to have all the HLA class I and HLA class II genes deleted. Rather than both copies of the gene being deleted, which is somewhat uncommon, cancer genomes will often have deleted one allele of a given gene, i.e., loss of heterozygosity (LOH).

HLA LOH causes cancer cells to lack expression of peptide-MHC on the surface membrane. In consequence, it is a resistance mechanism against TCR-based or peptide-MHC-based immunotherapies. These immunotherapies include immune checkpoint inhibition (ICI) also known as immune checkpoint blockade (ICB), engineered TCR-T cell therapy, tumor infiltrating lymphocyte (TIL) therapy, cytokine therapy (an example would be interleukin-2 also known as IL-2), T-cell engager therapy (an example would be ImmTACs), and cancer vaccines.

There are no predictive liquid biopsy assays on the oncology market that detect HLA LOH for predicting resistance to the immunotherapies listed above. Existing liquid biopsy diagnostic tests include an assay for detecting tumor mutation burden high (TMB-H) status in cancers, which helps in selecting patients for ICI but has been shown to be insufficient for predicting resistance/response compared to also using HLA LOH as a biomarker. Another liquid biopsy test predicts response to small molecule targeted inhibitor drugs for lung cancer based on mutations. There is also software for HLA LOH detection in tumors, but not for liquid biopsy applications. Thus, there is a need in the art for methods to determine HLA LOH status via liquid biopsy.

### SUMMARY OF THE INVENTION

Provided herein is a method of detecting human leukocyte antigen (HLA) gene loss of heterozygosity (LOH) in a subject. The method may comprise providing biopsy HLA sequence data, which may be obtained from cell-free DNA (cfDNA) of a liquid biopsy sample from the subject. The subject may have cancer. The cfDNA may comprise cancer cell DNA. The HLA sequence data may comprise sequences of one or more HLA genes, whole genome sequence (WGS) data, or whole exome sequence (WES) data.

The method may comprise providing germline HLA sequence data, which may be obtained from DNA of a germline cell sample of the subject. The germline cell sample may comprise peripheral blood monocytes (PBMC). The germline HLA sequence data may comprise sequences of one or more HLA genes, WGS data, or WES data. Each of one or more HLA genes from the germline cell DNA may be genotyped to two-, four, six-, or eight-digit resolution.

The method may comprise aligning coding sequences of both alleles of each HLA gene from the germline cell DNA to a reference sequence for each HLA gene to generate an alignment, and aligning cfDNA coding sequences to a reference genome so that reads at heterozygous bases can be counted. The method may comprise, from the alignment, identifying heterozygous bases of each HLA gene. Each heterozygous base may be identified by coding sequence position and genome coordinate. The method may comprise, for each heterozygous base of each HLA gene, counting allelic reads in the biopsy HLA sequence data and counting allelic reads in the germline HLA sequence data.

The method may comprise calculating a weight for each heterozygous base. The weight calculation may comprise, separately for allelic reads in the biopsy HLA sequence data and the germline HLA sequence data, for each heterozygous base of each HLA gene, dividing the read count of each heterozygous base by the sum of read counts on all heterozygous bases. The weight calculation may also comprise calculating the average at each heterozygous base between the biopsy HLA sequence data and the germline HLA sequence data.

The method may comprise comparing the allelic read count from the biopsy HLA sequence data to the allelic read count from the germline HLA sequence data, which may be by performing a Student's t-test with paired observation for each heterozygous base, in which each weight is multiplied by an observation comprising the read count at the heterozygous base in the biopsy HLA sequence data and the read count at the same heterozygous base in the germline DNA sequence data, to generate a p-value for each HLA gene. A significant p-value may be indicative of LOH for a HLA gene in the liquid biopsy. The significant p-value may be 0.004.

Also provided herein is a method of predicting a subject's response to a cancer treatment that targets an antigen bound to a restriction element encoded by a HLA gene. The method may comprise detecting LOH for the HLA gene according to the method of detecting human leukocyte antigen (HLA) gene loss of heterozygosity (LOH) in a subject described herein. Presence of LOH for the HLA gene may be indicative that the subject will have a poor response to the cancer treatment. Absence of LOH for the HLA gene may be indicative that the subject will respond or is more likely to respond to the cancer treatment, which may be as compared to an individual who has LOH for the HLA gene. The cancer treatment may be a T cell receptor (TCR)-based therapy. The TCR-based therapy may be an immune checkpoint blockade, a T cell engager, a TCR-T cell therapy, tumor infiltrating lymphocytes, a cancer vaccine, or a cytokine therapy. The T cell engager may comprise immune mobilizing TCRs against cancer.

Further provided herein is a method of treating cancer in a subject in need thereof, which may comprise administering to the subject a cancer treatment that targets an antigen bound to a restriction element encoded by a HLA gene; use of the cancer treatment in the manufacture of a medicament for treating cancer; and the cancer treatment for use in treating cancer. The subject may have been predicted to respond or be more likely to respond to the cancer treatment according to the method of predicting a subject's response to a cancer treatment that targets an antigen bound to a restriction element encoded by a HLA gene as described herein. The cancer treatment may be a T cell receptor (TCR)-based therapy. The TCR-based therapy may be an immune checkpoint blockade, a T cell engager, a TCR-T cell therapy, tumor infiltrating lymphocytes, a cancer vaccine, or a cytokine therapy. The T cell engager may comprise immune mobilizing TCRs against cancer.

Provided herein is a system for detecting HLA LOH in a subject. The system may comprise a computing system including a processor in communication with a memory. The memory may include instructions, which, when executed, may cause the processor to perform one or more steps of the method of predicting a subject's response to a cancer treatment that targets an antigen bound to a restriction element encoded by a HLA gene as described herein. Further provided herein is a system for predicting a subject's response to a cancer treatment that targets an antigen bound to a restriction element encoded by a HLA gene. The system may comprise a computing system, which may include a processor in communication with a memory. The memory may include instructions, which, when executed, cause the processor to detect LOH for the HLA gene as described herein. Also provided herein is a system of treating a cancer in a subject in need thereof. The system may comprise a computing system. The computing system may include a processor in communication with a memory. The memory may include instructions, which when executed, cause the processor to instruct that the subject be administered a cancer that targets an antigen bound to a restriction element encoded by a HLA gene. The subject may have been predicted by the processor to respond to the cancer treatment according to a system described herein.

### BRIEF DESCRIPTION OF THE DRAWINGS

FIG. 1 shows HLA gene locations on the p arm of Chromosome 6. The classical HLA class I genes are HLA-A, HLA-B, and HLA-C, highlighted in black; the HLA class II genes that encode MHC beta chains are HLA-DPB1, HLA-DQB1, and HLA-DRB1, also highlighted in black. Non-classical HLA class I genes and the HLA class II genes that encode an alpha chain are highlighted in grey. Note that for class II HLA genes, the MHC proteins have both an alpha and beta chain; for HLA class I genes, the MHC only has an alpha chain and that molecule binds to a beta-2-microglobulin molecule to form the MHC-peptide presentation unit. tel=telomere, cent=centromere.

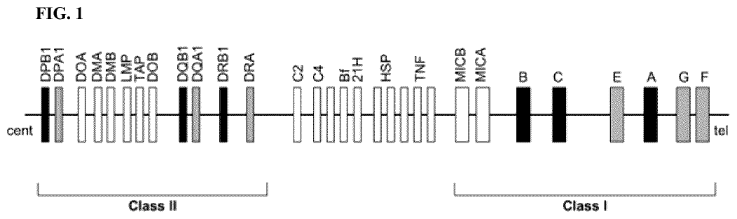

FIG. 2 shows an overview of a method of detecting HLA LOH via a liquid biopsy as described herein. Cell-free DNA (cfDNA) is extracted from plasma which is extracted from a blood sample from a cancer patient. The cfDNA is sequenced (in one embodiment, germline DNA from the same patient is also sequenced as a normal DNA control), and a method is utilized for detecting HLA LOH if it is present in the patient's cancer. HLA LOH can cause resistance to TCR-based immunotherapies such as those listed in pink. If a patient has HLA LOH in their cancer, one of the therapy options unaffected by HLA LOH (left side) can be utilized for treating the patient.

FIG. 3 shows an example text file showing the coding sequence alignment between a reference HLA-A*03:01:01:01 coding sequence (SEQ ID NO: 1) and two HLA-A alleles from a patient.

FIG. 4 shows an example heterozygous base table with read counts and calculated frequencies for both alleles.

FIG. 5 shows reads mapped to HLA-A from tumor DNA, liquid biopsy cfDNA, and germline DNA from two patients.

FIG. 6 shows a potential usage of the method described herein in an assay that may be classified as a clinical trial assay (CTA) that helps determine enrollment on clinical studies for TCR-T cell therapies.

FIG. 7 shows the results of statistical analyses that were performed to determine how many test cases would be required to ensure the sensitivity and specificity of an HLA LOH detection method described herein to a given level of confidence.

FIG. 8 is an exemplary computer system for effectuating the methods of determining loss of heterozygosity in human leukocyte antigen in liquid biopsies illustrated in FIG. 2.

### DETAILED DESCRIPTION

The inventors have developed methods for detecting human leukocyte antigen (HLA) loss of heterozygosity (LOH) via liquid biopsy, which can be used to determine whether a subject's cancer exhibits HLA LOH. This method provides a substantial advantage because is allows for HLA LOH status to be determined before selecting a treatment for a patient who is a candidate for cancer immunotherapy. Approximately 44% of all cancer patients in the United States are eligible for FDA-approved immune checkpoint inhibition (ICI) therapy alone. However, there is a low efficacy rate (13%) for ICIs, despite some promising clinical results and a relatively low occurrence of side-effects. Furthermore, 17% of all cancer patients have HLA LOH in their tumors, which could account for a portion of the patients in which there is a lack of efficacy; the efficacy of ICI could effectively double if patients who have HLA LOH are not treated with ICI but instead a different treatment type that may be more effective for those patients. Some cancers have as high as a 42% prevalence of HLA LOH in patients (e.g., thymic cancer). Patients with these types of cancers may perhaps benefit the most from a test that can detect HLA LOH pre-treatment.

Another type of immunotherapeutic that is affected by HLA LOH is T cell receptor (TCR)-T cell therapy. Although still in clinical trials, TCR-T cell therapies have shown promise in patients, resulting in tumor regression for a portion of patients. HLA LOH has been demonstrated to be a resistance mechanism for this type of therapy. In this case, the therapy involves an engineered TCR that targets one specific HLA gene and allele, for example, HLA-A*01:01:01:01 which is targeted by KK-LC-1-TCR-T cell therapy (being tested against gastric, cervical, lung, breast, and other epithelial cancers). If a patient's cancer deletes the HLA gene and allele that the TCR-T cell therapy is targeting, the therapy will theoretically have no chance of working because there will be no molecule for the T cells to bind to on the surface of the cancer cells. Thus, it would be useful to determine whether a patient's cancer harbors HLA LOH, before a patient is treated with this type of therapy. For the other therapies listed as being impacted by HLA LOH, resistance would be expected for the same reason-no MHC-peptide complex expressed on the cancer cell surface to which T cells or T-cell engagers would normally bind.

If HLA LOH is detected in a patient's cancer before treatment selection, this can save the patient's life by allowing for selecting a treatment that is more likely to help the patient rather than treating the patient with a therapy that is unlikely to work because of the HLA LOH. Furthermore, selecting a treatment utilizing this precision oncology approach will help the patient avoid toxic side-effects from any treatments that will not help them. An additional advantage is cost savings: the patient, their family, and insurance companies will save money by avoiding treatment with a therapy that will not work for the patient. Since some immunotherapies are quite expensive, in the range of $500,000 per patient, the cost savings could be substantial.

It would be advantageous to test for HLA LOH using a non-invasive method. Rather than surgically extracting solid tumor tissue for a biopsy to detect HLA LOH status, a non-invasive method such as liquid biopsy would allow for testing the biomarker status without the patient having to undergo surgery. This is helpful in several ways, including less pain for patients, lower cost to obtain a sample, and easier processing of the sample. Further, a liquid biopsy test may return more accurate results than an HLA LOH test using tumor tissue and allow for sampling across a patient's tumors and tumor regions rather than taking a solid tumor tissue sample representative of only one region of one tumor. For example, if a solid tissue region of one tumor from a patient returns a negative HLA LOH test result, there is no guarantee that another region of the same tumor or another tumor does not harbor HLA LOH. With a liquid biopsy sample, information is obtained across tumors and tumor regions from a patient, allowing for detection of HLA LOH in any one of these tumors and/or regions.

The inventors developed methods described herein to detect HLA LOH via liquid biopsy. Due to the highly polymorphic nature of the HLA genes, it is a difficult problem to detect HLA LOH in tumor tissue or in a liquid biopsy sample. In fact, the TCGA publicly available copy number variant data in cBioPortal does not include the HLA genes, while it does include most other ˜20,000 protein-coding genes. Despite software programs that aim to detect HLA LOH in tumors, there are no methods designed and tested for doing so in liquid biopsies.

The method will be described in more detail below. In short, in one embodiment of the method, cell-free DNA (cfDNA) may be extracted from blood plasma and sequenced. Technologies for cfDNA extraction and sequencing are advantageous in that they are ahead of other methodologies that could potentially be utilized (and will be described below). However, in liquid biopsy methods that involve cfDNA extraction and sequencing, the highly polymorphic nature of the HLA region is further complicated by the fact that there is cfDNA from not just tumor cells but also from healthy cells. The method described deals with this complication so that tumor-derived HLA LOH can be detected via cfDNA. The inventors have unexpectedly discovered that HLA LOH can be detected from cfDNA in a liquid biopsy.

The method described herein is not only limited to LOH of HLA class I genes, but may also be used to detect LOH of HLA class II genes. Both HLA class I LOH and HLA class II LOH may be relevant biomarkers for predicting resistance to immunotherapies in cancer patients, as both CD8+ and CD4+ T cells have been shown to be important in TCR-based immunotherapy efficacy.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

### 2. Detecting Loss of Heterozygosity in HLA

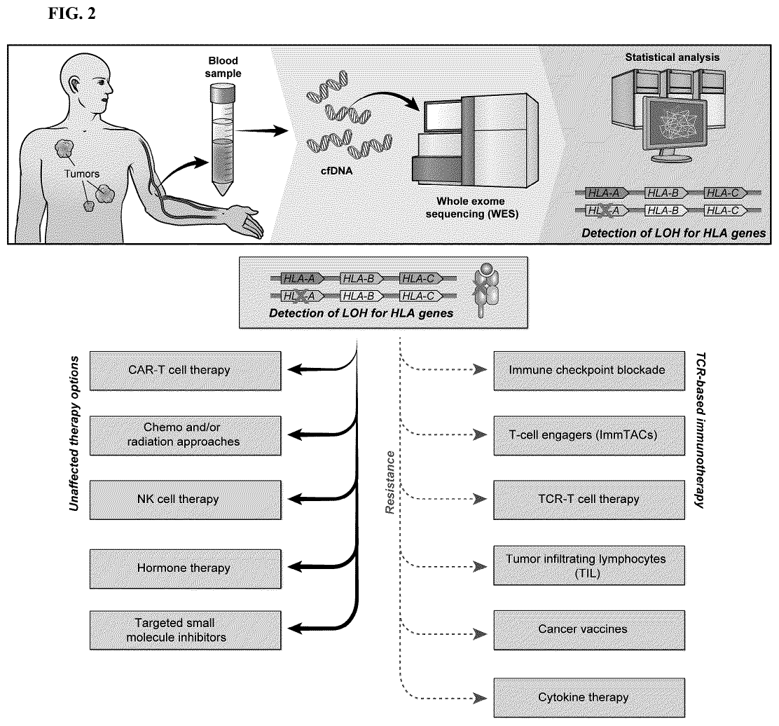

Provided herein is a method of detecting HLA LOH in a subject. A schematic of one example of the method is provided in FIG. 2. In one example, the subject has a cancer. The method may comprise performing a liquid biopsy.

a. Sample Collection

The method may comprise obtaining or providing a sample from the subject. The sample used in the liquid biopsy may comprise cell-free DNA (cfDNA). The sample may be blood, urine sample, saliva, cerebrospinal fluid, stool, or any type of bodily fluid that contains enough DNA from tumors, cells from tumors, or cellular components from tumors. In one example, the sample is blood. The blood sample may comprise plasma, which may be extracted from the blood. In another example, the sample comprises circulating tumor cells (CTC), which may be extracted from the blood sample. The CTC may be extracted from a buffy coat of the blood. In a further example, the sample comprises extracellular vesicles, which may be extracted from a blood sample or a urine sample.

The liquid biopsy sample may be collected from a subject by any method that preserves the cfDNA, CTCs, EVs, or any component that is utilized for the HLA LOH detection. Blood may be collected in any way that is consistent with accepted medical standards that will minimize harm and pain to the subject. In one example, the blood sample is collected through a venous blood draw. Blood and plasma samples may be collected into tubes that maintain the integrity of the cfDNA, CTCs, EVs or other components utilized for the HLA LOH detection method. For example, Streck tubes can keep plasma at room temperature for 5-10 days and preserve the cfDNA. EDTA tubes have been utilized for collecting blood as well. Any tube that preserves the integrity of the sample for HLA LOH detection can be utilized. Similarly for sample storage, samples must be stored in such a way that preserves the integrity of the sample. This depends on the collection and storage tubes utilized for a given sample.

The method described herein may comprise providing or obtaining cfDNA DNA. Cell-free DNA may be extracted using any technique known in the art. In one example, cfDNA is extracted from plasma using the QIAMP CIRCULATING NUCLEIC ACID KIT. Other embodiments may use different kits and/or techniques for cfDNA extraction. In one example, at least 20 ng cfDNA is extracted for whole exome sequencing (WES). Cell-free DNA sequences may be obtained or provided from cfDNA extracted from CTCs; polymerase chain reaction (PCR) (e.g., digital PCR) on cfDNA; WES on CTC DNA; PCR on CTC DNA; whole genome sequencing (WGS) on cfDNA; WGS on CTCs; WES on EVs; PCR on EVs; WGS on EVs; or any other method that involves determining sequence and read count information from the HLA region of the genome of cancer cells from a given patient.

The method described herein optionally comprises providing or obtaining germline DNA from the subject. Germline DNA may be extracted from peripheral blood mononuclear cells (PBMC), which may be extracted from a blood sample. The germline DNA may also be extracted from a non-malignant tissue from the patient, which may be normal tissue adjacent to a tumor. The germline DNA may be sequenced, and the sequence may be used as a germline control for detecting HLA LOH. The germline DNA may be extracted using standard methods for DNA extraction, which are known in the art. In one example, at least 1 million PBMCs are obtained or provided for germline DNA extraction for use as a control.

b. Sequencing

The method described herein comprises obtaining or providing sequences from cell-free DNA (biopsy HLA sequence data) and optionally germline DNA (germline HLA sequence data) from the subject. In one example, the sequencing comprises WES. The cfDNA may be from plasma and the germline DNA may be from PBMC. In another example, the sequencing comprises at least one of targeted sequencing and whole genome sequencing (WGS). The sequencing may comprise a PCR-based method, which may be digital PCR or another PCR-based method.

The sequencing may comprise performing standard library preparation from the cfDNA and optionally the germline DNA. The sequencing may also comprise standard exome capture. In an alternative embodiment, custom capture kits may be used or created for more sensitively capturing a patient's HLA DNA from the sample before sequencing.

The WES may be performed using any known method known in the art, which may be using an ILLUMINA sequencer. Fastq files may be generated from the sequencing. In an alternative embodiment in which PCR-based methods are used for HLA LOH detection, the output files from PCR may be a different file format. Also in an alternative embodiment, sequencing could be performed on a different sequencer or type of sequencer and at a different sequencing depth, as well as using different library prep.

In one example, fastq files are aligned to the hg38 reference genome using the BWA alignment tool, although other alignment tools may also be used. The output of alignment may comprise a bam file for each sample. For each subject, there may be a pair of bam files-one for the cfDNA and one for a matched germline sample. Alternative embodiments to this step are possible, including one where all reads are removed except for HLA-aligned reads and possibly unmapped reads, and all remaining reads are re-mapped only to the HLA genes.

c. HLA Typing

The method may comprise HLA typing. In one example, germline sequencing data, which may be in a bam file or fastq file, may be used. In one example, a standard HLA typing method is performed, which may be at least one of OptiType and Polysolver. HLA may be typed at 2-, 4-, 6-, or 8-digit resolution, which may be defined by the coding (exonic, also known as complementary or cDNA) DNA sequence of each HLA allele. In one example, HLA is typed to 6-digit resolution. This is as opposed to 2-digit resolution or serotype of the allele, 4-digit resolution which is the amino acid sequence of the allele, and 8-digit typing which is the nucleic acid sequence of the allele including its introns. Six-digit HLA typing resolution may be sufficient because WES data correspond to the exonic regions of each HLA gene, which are defined by the 6-digit typing resolution. In another example, other levels of resolution for HLA typing and other HLA typing methods may be used, for example, methods that utilize microarrays. The HLA LOH detection method may be directed to HLA genes implicated in a specific cancer, such as for leukemia. In one example, HLA typing is performed to 8-digits, whereby intronic sequence regions are utilized for HLA typing and determining HLA LOH for the bone marrow transplantation relapse context. In one example, all HLA class I genes are genotyped for a given patient, but in other embodiments HLA class II genes may also be genotyped for a given patient. In one example, all HLA genes that are being tested for LOH are genotyped for a given patient

d. Identifying Heterozygous Bases

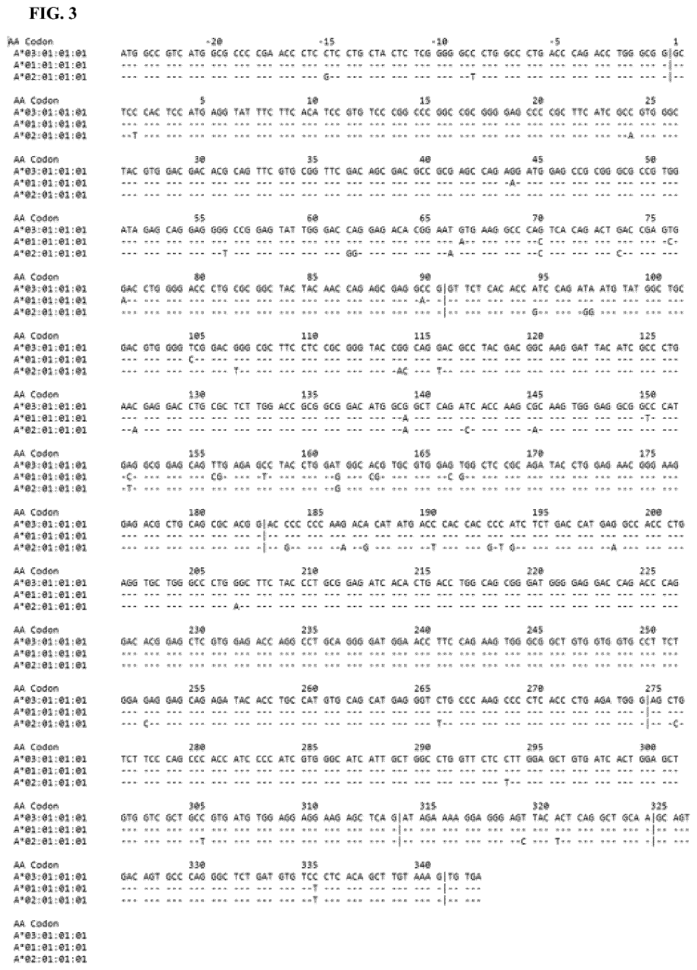

The method may comprise identifying heterozygous bases in HLA genes, which may be by performing an alignment of each HLA gene sequence from the subject to one or more known HLA gene sequences. In one example, the Immuno Polymorphism Database (IPD)-ImMunoGeneTics project (IMGT)/HLA web-based alignment tool at www.ebi.ac.uk/ipd/imgt/hla/alignment/may be utilized for querying the coding sequence of each subject's HLA gene, which may be one or more of HLA-A, HLA-B, and HLA-C genes. A different source for the HLA gene sequence information may be used. In one example, the alignment outputs text that can be placed into a text file, and a setting may be chosen so that only the bases that are different from the reference allele are shown as letters in the text; the other bases are shown as horizontal dashes (FIG. 3). When generating the sequence information for each allele, the alleles for a given patient may be determined from the HLA typing step described above.

e. Counting Reads at Heterozygous Bases

The method may also comprise counting reads at heterozygous bases. In one example, the read counts at heterozygous bases are compared for both alleles between the biopsy DNA sample and the germline DNA sample, to determine if there is an HLA LOH event in the subject's cancer. One embodiment, the allelic read frequency is calculated at each heterozygous base, and the read frequencies for one allele in the biopsy DNA sample are compared to the read frequencies from that same allele in the subject's germline DNA sample. Therefore, the first step after identifying the heterozygous bases for a given subject's alleles for a given HLA gene may be to count the reads at each heterozygous base for that subject and gene. In another example, the reads at each heterozygous base are counted, but the frequencies are not calculated.

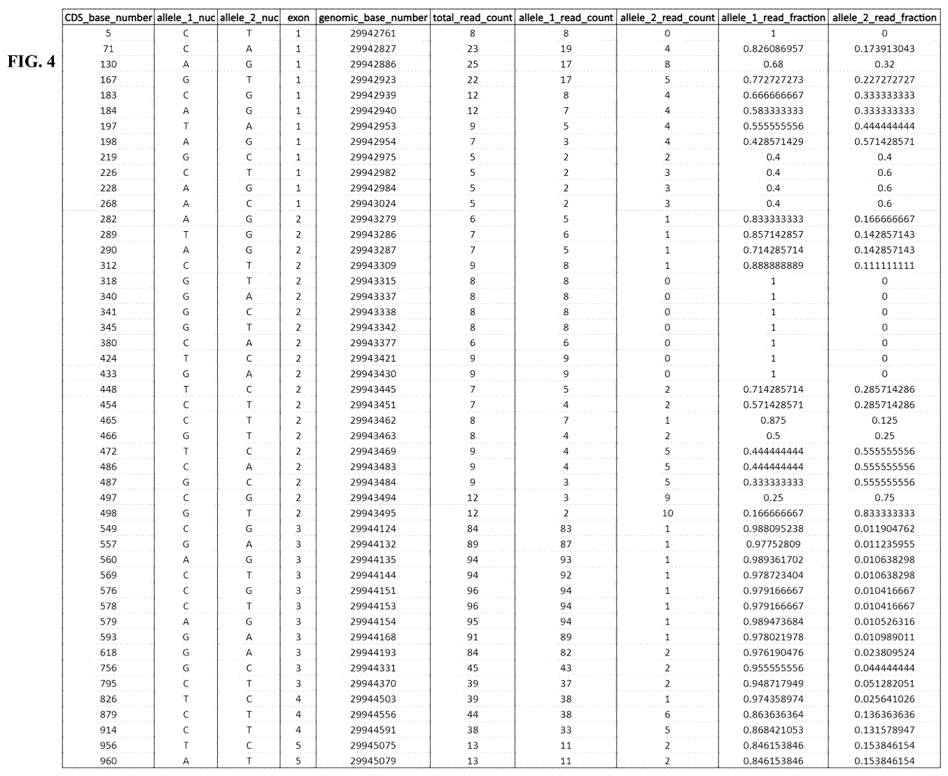

In one example, R programming language is used to implement a method to read the alignment text file from IPD-IMGT/HLA and generate a heterozygous base table, such as one shown in FIG. 4. The table may include the read counts and calculated read frequencies at each heterozygous base for that HLA gene and patient. In other examples, a table with a different format is generated, the table generation step is skipped, a different programming language is used to generate the table, and/or different code is used to generate the table.

As shown in FIG. 4, a heterozygous base table with read counts and calculated frequencies for both alleles may be generated. In one example, two such tables are generated for each HLA gene (HLA-A, HLA-B, HLA-C) separately for a given subject: one table for that gene in the biopsy DNA sample from that subject and one table for that gene in the germline DNA sample from that subject. Read frequencies may be calculated for both allele 1 and allele 2. In another example, read fraction is calculated for only one allele, or read frequencies are not explicitly calculated at all.

To count reads at heterozygous bases, an R script may be used. The R program may read a “read count” file that may be generated using the IGVTools Counts tool. The IGVTools Counts tool may returns a file, which may be a .wig file, that has a table showing how many reads were counted from the sequencing experiment at each base in the selected region of the genome. In one example, the region of Chromosome 6 that contains each HLA class I gene, HLA-A, HLA-B, and HLA-C is selected, and a separate .wig file is generated for each HLA gene from each patient for both the biopsy DNA sample and the germline DNA sample from the subject. A different tool for outputting read count information may be used. In one example, the read count information is determined from the bam file that was generated in the earlier alignment step from the fastq files. This bam file may be used as input to IGVTools Counts and the .wig file may be generated with the read counts information at each base in the region of each HLA gene. The table from the IGVTools Counts output may then be utilized as input to the R program, which may determine the read count at each heterozygous base for both alleles. Different code may be used to read the read counts information and incorporate it into the determination of HLA LOH.

f. Calculating Weights for the Statistical Test to Determine HLA LOH Status

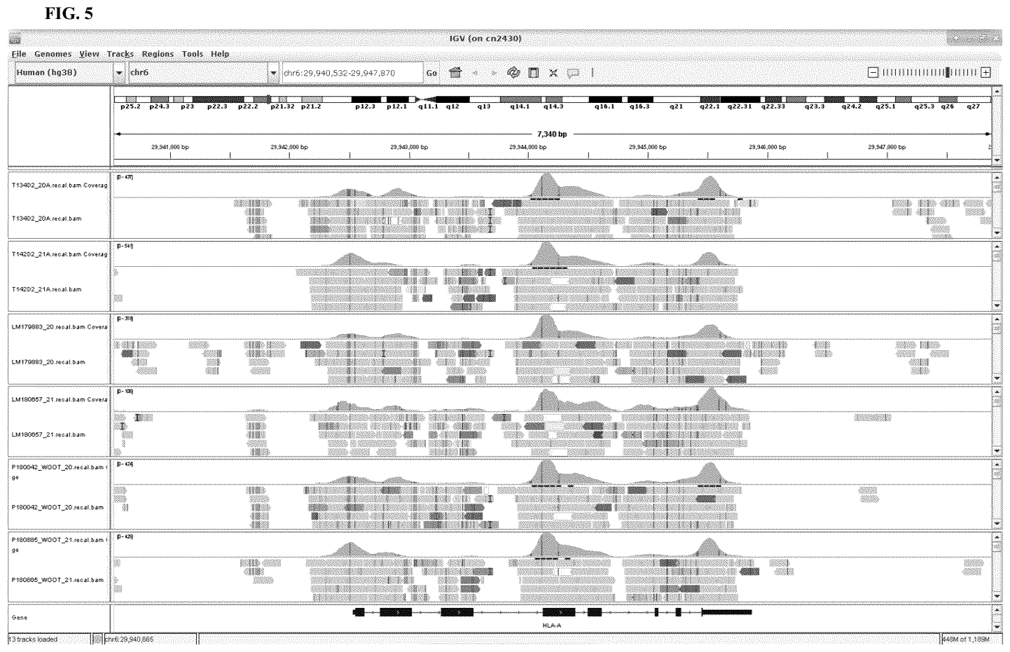

The method may comprise calculating weights for a statistical test to determine HLA LOH status. To capture the biological and experimental system into a statistical model for calculating an indicator of HLA LOH, weights may be utilized in the calculation. The weights may reflect the fact that in a WES experiment, some heterozygous bases will accrue more reads than others in the same gene and patient due to exome capture and alignment (FIG. 5).

In one example as shown in FIG. 5, reads are mapped to HLA-A from tumor DNA, liquid biopsy cfDNA, and germline DNA from two patients. The mapping may show that the phenomenon of unbalanced read stacking occurs in germline, liquid biopsy, and tumor samples, which may need to be addressed in some embodiments of the liquid biopsy test. This may be caused by exome capture and alignment. This phenomenon may be addressed in different ways, such as customized capture kits and alignment algorithms.

The weights may be calculated as follows. A weight may be calculated for each heterozygous base for a given HLA gene and subject. For example, one subject's HLA-A heterozygous bases may be different than another subject's HLA-A heterozygous bases, and each subject and HLA gene may have a different number of heterozygous bases.

To calculate the vector of weights for a given patient and HLA gene, each heterozygous base's read count may be divided by the sum of read counts on all the heterozygous bases, and this may be separately calculated for the biopsy DNA sequence data and the germline DNA sequence data. The average at each heterozygous base between the biopsy DNA and the germline DNA for a given HLA gene and subject may be calculated as follows resulting in the weights, where the number of weights (that is, the length of the weight vector) is equal to the number of heterozygous bases for a given HLA gene for a given patient:

weight_vector=[vector_of_plasma_read_counts_at_heterozygous_bases/sum(vector_of_plasma_read_counts_at_heterozygous_bases)

+

vector_of_normal_read_counts_at_heterozygous_bases/sum(vector_of_normal_read_counts_at_heterozygous_bases)]/2

In another example, the weights may be calculated by taking the average read count at each heterozygous base between the biopsy sample and normal sample for a given HLA gene and subject and then summing those averages and dividing each average by the sum to obtain the weights, where, again, the length of the weight vector is equal to the number of heterozygous bases for a given HLA gene and patient:

weight_vector=[(vector_of_plasma_read_counts_at_heterozygous_bases+vector_of_normal_read_counts_at_heterozygous_bases)/2]/sum[(vector_of_plasma_read_counts_at_heterozygous_bases+vector_of_normal_read_counts_at_heterozygous_bases)/2]

The weights may be calculated a different way as opposed to how they are calculated in the two examples above, or weights may not be used at all.

g. Statistical Test for Determining HLA LOH in a Liquid Biopsy Sample

The method may comprise performing a statistical test to detect HLA LOH. A statistical test involving the weights described above may be run, and may return a numerical value that indicates HLA LOH positive or negative status. In one example, a Student's t-test with paired observations and with the weights multiplied by the observations may be performed. In another example, one or more other statistical tests may be used, which may or may not use weights. In one example where weights are used, the weights are calculated in different ways.

The Student's t-test may be used to calculate a p-value that determines whether one group of observations is significantly different from another group of observations. The Student's t-test may be appropriate because each observation is the read frequency at a heterozygous base. For a given subject and HLA gene, the heterozygous base position is the same genomic location for both the biopsy sample and germline sample. The paired observations are therefore the read count at a given heterozygous base for the biopsy sample and the read count at the same heterozygous base for the normal sample from the same patient. The p-value determines if, across the heterozygous bases for that gene and that patient, there is a significant difference between read frequencies for a given allele. The read frequency at one heterozygous base is the read count at that base for that allele divided by the total read count at that heterozygous base from both alleles from that sample. If the read frequencies of that allele are significantly different in the biopsy DNA compared to the germline DNA, then an HLA LOH is called and HLA LOH status is positive. If the read frequencies are not significantly different, then HLA LOH status is negative. This can be done separately for both alleles, to get a p-value for each allele. In other embodiments, the read counts, rather than the read frequencies, may be used to compare biopsy DNA sample to germline DNA sample for a given patient and HLA gene. A method that does not involve comparing the biopsy DNA sample to germline DNA sample may be used. Such a method may only use information from the biopsy DNA sample.

In one example, the weights are multiplied by the frequency observations in a dot-product fashion and then the paired t-test is run, which may be in the R programming language, and may utilize built-in R functions or user-defined functions. In other embodiments, the same test may be in a different programming language, using different code, or may use a totally different test and also implement that in R or in a different programming language or with different code. The output of a test for each allele may be a p-value. The p-value cutoff may be 0.004. In other embodiments, the p-value cutoff may be 0.05, 0.01, 0.005, or 0.001. The p-value cutoff may be determined from testing the predictive method on samples and identifying the cutoff value that best distinguishes positive calls from negative calls to maximize the number of true positives and true negatives and minimizes the number of false positives and false negatives. The p-value cutoff may depend on the statistical test that is used. The p-value may be any value between 0 and 1 that best separates positive calls from negative calls.

If the p-value is lower than this for both alleles, then HLA LOH status may be deemed positive, and if at least one of the allelic p-values is higher, then the HLA LOH status may be deemed negative. The test may also determine which allele, if any, was lost in the HLA LOH event by observing the sign of the difference of weighted averages computed within the t-test. In another embodiment, the difference may be of unweighted quantities if no weights are used. In a further embodiment, another quantity may be utilized rather than the averages. In other embodiments, which allele was lost may be determined by observing a sign of a parameter calculated as part of the test used in that alternative embodiment, or by some other type of indicator.

HLA LOH in the subject may be indicative of resistance or responsiveness to one or more cancer immunotherapies. The cancer immunotherapy may be a cancer treatment that targets an antigen bound to a restriction element encoded by a HLA gene. If the subject exhibits HLA LOH, then subject may be predicted to be a poor responder to, and thus may not be a candidate for, one or more cancer treatments, which may be one or more T cell receptor (TCR)-based immunotherapies. The TCR-based immunotherapy may comprise one or more of an immune checkpoint blockade (e.g., but not limited to, Pembrolizumab, avelumab, ipilimumab, nivolumab; a T cell engager, which may be an immune mobilizing monoclonal TCRs against cancer; a T cell receptor-T cell therapy (e.g., but not limited to, one targeting KK-LC-1, HPV-16 E7, GP100, NY-ESO-1); tumor-infiltrating lymphocytes (TIL); a cancer vaccine (e.g., but not limited to, Sipuleucel-T); and cytokine therapy (e.g., but not limited to, interleukin-2). If the subject does not exhibit HLA LOH, then the subject may be predicted to more likely be responsive to one or more TCR-based immunotherapies.

### 3. Cancer Treatments

Provided herein is a method of treating cancer in a subject. The method may comprise administering a cancer treatment to the subject. Loss of heterozygosity may have been detected in one or more HLA genes of the subject as described herein. In one example, if HLA LOH has been detected in the subject, then a TCR-based immunotherapy may not be administered to the subject, and instead one or more of a CAR-T cell therapy, chemotherapy, radiation therapy, natural killer (NK) cell therapy, hormone therapy, and a targeted small molecule inhibitor may be administered to the subject. If HLA LOH has not been detected in the subject, then a TCR-based immunotherapy may be administered to the subject. The TCR-based therapy may be one or more of immune checkpoint blockade, a T cell engager, a TCR-T cell therapy, tumor infiltrating lymphocytes, a cancer vaccine, and a cytokine therapy. Examples of immune checkpoint blockade therapy include Pembrolizumab (FDA-approved for multiple cancer types), Atezolizumab, Ipilimumab, Avelumab, and Nivolumab. Examples of T-cell engagers include KIMMTRAK, which is FDA-approved for treating uveal melanoma. Examples of TCR-T cell therapies include KK-LC-1-TCR-T cells, HPV-16 E7-TCR-T cells, gp100-TCR-T cells, and NY-ESO-1-TCR-T cells, all of which are being tested in clinical trials. Examples of TIL therapies include Lifileucel. Examples of cancer vaccines include Sipuleucel-T, which is FDA-approved for treating asymptomatic or minimally symptomatic metastatic castration-resistant prostate cancer (mCRPC). Cytokine therapies include Interleukin-2.

### 4. System

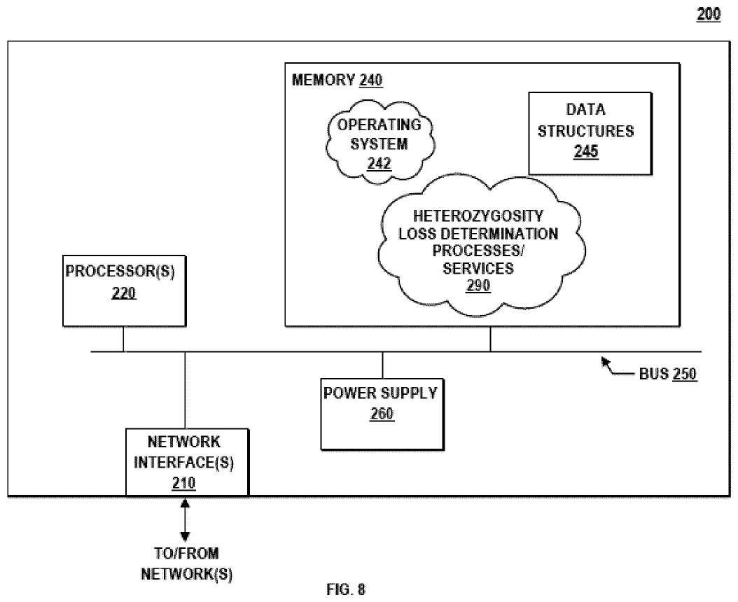

FIG. 8 is a schematic block diagram of an example device 200 that may be used with one or more embodiments described herein, e.g., as a component of the system for determining heterozygosity loss in human leukocyte antigen in liquid biopsies.

Device 200 comprises one or more network interfaces 210 (e.g., wired, wireless, PLC, etc.), at least one processor 220, and a memory 240 interconnected by a system bus 250, as well as a power supply 260 (e.g., battery, plug-in, etc.).

Network interface(s) 210 include the mechanical, electrical, and signaling circuitry for communicating data over the communication links coupled to a communication network. Network interfaces 210 are configured to transmit and/or receive data using a variety of different communication protocols. As illustrated, the box representing network interfaces 210 is shown for simplicity, and it is appreciated that such interfaces may represent different types of network connections such as wireless and wired (physical) connections. Network interfaces 210 are shown separately from power supply 260, however it is appreciated that the interfaces that support PLC protocols may communicate through power supply 260 and/or may be an integral component coupled to power supply 260.

Memory 240 includes a plurality of storage locations that are addressable by processor 220 and network interfaces 210 for storing software programs and data structures associated with the embodiments described herein. In some embodiments, device 200 may have limited memory or no memory (e.g., no memory for storage other than for programs/processes operating on the device and associated caches). Memory 240 can include instructions executable by the processor 220 that, when executed by the processor 220, cause the processor 220 to implement aspects of the system 100 and associated methods outlined herein.

Processor 220 comprises hardware elements or logic adapted to execute the software programs (e.g., instructions) and manipulate data structures 245. An operating system 242, portions of which are typically resident in memory 240 and executed by the processor, functionally organizes device 200 by, inter alia, invoking operations in support of software processes and/or services executing on the device. These software processes and/or services may include heterozygosity loss determination processes/services 290, which can include aspects of the methods and/or implementations of various modules described herein. Note that while the heterozygosity loss determination processes/services 290 is illustrated in centralized memory 240, alternative embodiments provide for the process to be operated within the network interfaces 210, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the heterozygosity loss determination processes/services 290 is shown as a standalone process, those skilled in the art will appreciate that this process may be executed as a routine or module within other processes.

The present invention has multiple aspects, illustrated by the following non-limiting examples.

### Example 1

### Detecting HLA Loss of Heterozygosity

This example demonstrates a method of detecting HLA LOH via liquid biopsy.

### Methods

### Sample Collection and Storage

Ten mL blood samples were collected via venous blood draws from each patient and plasma was extracted using standard protocols. Plasma volume of 1 mL was enough plasma to contain enough cfDNA for the HLA LOH detection experiments. In addition, PBMCs were collected utilized as a germline control.

cfDNA Extraction

Cell-free DNA was extracted from plasma using a QIAMP CIRCULATING NUCLEIC ACID KIT. At least 20 ng cfDNA was extracted for whole exome sequencing (WES). Germline DNA was also extracted from PBMCs using a standard method for DNA extraction.

### Sequencing

WES was performed on the cfDNA extracted from plasma and on the germline DNA extracted from PBMCs from the same patients from whom cfDNA was extracted. Standard library prep was performed, as well as standard exome capture. No custom kits were used for HLA capture. Fastq files were generated from the WES and aligned to the hg38 reference genome using the BWA alignment tool. The output of alignment was a bam file for each sample. For each patient, there was a pair of bam files: one for the cfDNA and one for the matched germline sample.

### HLA Typing

Germline sequencing data (as a bam file or fastq file) were utilized for HLA typing. Six-digit resolution was determined for HLA typing, which was defined by the coding (exonic, also known as complementary or cDNA) DNA sequence of each HLA allele.

### Finding Heterozygous Bases

The IPD-IMGT/HLA web-based alignment tool at www.ebi.ac.uk/ipd/imgt/hla/alignment/was utilized for querying the coding sequence of each patient's HLA-A, HLA-B, and HLA-C genes. The webtool text outputs were copied and pasted into a text file, and a setting was chosen so that only the bases that are different from the reference allele were shown as letters in the text; the other bases are shown as horizontal dashes (FIG. 3). When generating the sequence information for each allele, the alleles for a given patient are determined from the HLA typing step described above. The setting to only display exact allele matches in the webtool output was selected.

FIG. 3 is example text file showing the coding sequence alignment between two HLA-A alleles from a patient. HLA-A*03:01:01:01 was the allele used as reference, and this patient had HLA-A*01:01:01:01 and HLA-A*02:01:01:01 as HLA-A alleles, where the seventh and eighth digit of the allele are arbitrary because no matter what the seventh and eighth digit are, the coding sequence is the same. The vertical lines represent exon boundaries. The numbering shows the codons. Nucleotides are displayed in groups of 3, each representing an amino acid (AA) codon. Any base position where the patient's two alleles have a different nucleotide constitutes a heterozygous base.

### Counting Reads at Heterozygous Bases

The novel method presented here involves comparing the read counts at heterozygous bases for both alleles between the cfDNA sample and the germline normal DNA sample, to determine if there is an HLA LOH event in the patient's cancer. The allelic read frequency was counted at each heterozygous base, and the read frequencies for one allele in the cfDNA were compared to the read frequencies from that same allele in the patient's germline DNA sample. Therefore, the first step after identifying the heterozygous bases for a given patient's alleles for a given HLA gene was to count the reads at each heterozygous base for that patient and gene. A program in the R programming language was written to read the alignment text file from IPD-IMGT/HLA and generate a heterozygous base table like the one shown in FIG. 4.

The example table in FIG. 4 includes the read counts and calculated read frequencies at each heterozygous base for that HLA gene and patient. Two tables like this were generated for each HLA gene (HLA-A, HLA-B, HLA-C) separately for a given patient-one table for that gene in the cfDNA sample from that patient and one table for that gene in the germline control sample from that patient. Read frequencies were calculated for both allele 1 and allele 2.

To count reads at heterozygous bases, an R script was written to test the method in validation studies. The R program read a “read count” file that, in this embodiment, was generated using the IGVTools Counts tool. The IGVTools Counts tool returns a wig file that has a table showing how many reads were counted from the sequencing experiment at each base in the selected region of the genome. These settings were configured when running IGVTool Counts. The region of Chromosome 6 that contains each HLA class I gene, HLA-A, HLA-B, and HLA-C was selected. A separate .wig file was generated for each HLA gene from each patient for both the cfDNA sample and the germline normal sample from that patient. The read count information was determined from the bam file that was generated in the earlier alignment step from the fastq files. This bam file was used as input to IGVTools Counts and the .wig file was generated with the read counts information at each base in the region of each HLA gene. That table from the IGVTools Counts output was then utilized as input to the R program, which determined the read count at each heterozygous base for both alleles.

### Calculating Weights for the Statistical Test to Determine HLA LOH Status

To capture the biological and experimental system into a statistical model for calculating an indicator of HLA LOH, weights were designed for the calculation. The weights reflected the fact that in a WES experiment, some heterozygous bases will accrue more reads than others in the same gene and patient due to exome capture and alignment (FIG. 5).

The mapping in FIG. 5 shows that different locations along the gene had inherently different read counts. This was likely caused by exome capture and alignment. The weights were calculated as follows. There was a weight for each heterozygous base for a given HLA gene and patient. To calculate the vector of weights for a given patient and HLA gene, each heterozygous base's read count was divided by the sum of read counts on all the heterozygous bases. This was done separately for the cfDNA sample and the normal sample. Then the average at each heterozygous base between the cfDNA sample and the normal sample was calculated for a given HLA gene and patient, as described above.

### Statistical Test for Determining HLA LOH in a Liquid Biopsy Sample

With the weights calculated, the next step for determining HLA LOH status from liquid biopsies was to run a statistical test involving the weights that returns a numerical value that indicates HLA LOH positive or negative status. A Student's t-test was applied with paired observations and with the weights multiplied by the observations. The Student's t-test involved the calculation of a p-value that determines whether one group of observations is significantly different from another group of observations. A paired t-test was used because each observation was the read frequency at a heterozygous base; for a given patient and HLA gene, the heterozygous base position was the same genomic location for both the cfDNA sample and normal sample. The paired observations were therefore the read count at a given heterozygous base for the cfDNA sample and the read count at the same heterozygous base for the normal sample from the same patient. The p-value determined if, across the heterozygous bases for that gene and that patient, there was a significant difference between read frequencies for a given allele. The read frequency at one heterozygous base was the read count at that base for that allele divided by the total read count at that heterozygous base from both alleles from that sample. If the read frequencies of that allele were significantly different in the cfDNA compared to the normal, then an HLA LOH was called and HLA LOH status was positive. If the read frequencies were not significantly different, then HLA LOH status was negative.

The weights were multiplied by the frequency observations in a dot-product fashion (that is, weights•frequencies) and then the paired t-test was run in the R programming language using the built-in R function for running the t-test (i.e., t.test with the parameter paired=TRUE). The output of a test for each allele was a p-value, and the p-value cutoff was 0.004. If the p-value was lower than this for both alleles, HLA LOH status was deemed positive, and if at least one of the allelic p-values was higher than the HLA LOH status was deemed negative. The test was also able to determine which allele, if any, was lost in the HLA LOH event by observing the sign of the difference of averages computed within the t-test.

### Results

### Patients

The method described above was performed on 107 test cases. Each case corresponded to one HLA gene in one given patient. The 107 test cases were performed across 43 patients, broken down as follows: 2 HPV-negative head and neck cancer patients, 14 prostate cancer patients, and 27 breast cancer patients. The samples from the head and neck cancer patients were collected from patients on clinical trials at NCI and were sequenced at the NCI Genomics Core at Frederick National Laboratory; for the prostate cancer and breast cancer patients, WES data from their samples were downloaded from dbGaP (the database of Genotype and Phenotypes) and were obtained from an already conducted study.

### Samples

From each patient, whether from the NCI cohort or the dbGaP cohorts, there was WES data from DNA from three samples: a tumor sample, a liquid biopsy plasma cfDNA sample, and a germline control sample. The WES data were downloaded for the dbGaP cohorts, and were obtained by sequencing new samples for the NCI cohort. In all cases, DNA was extracted from tumors, plasma, and PBMCs from each patient. WES data were provided in the form of fastq files, and the data were utilized to test the described embodiment of the method on these samples.

Ground-Truth from the Head and Neck Cancer Cohort from NCI

HLA LOH results from running the above-described method on the liquid biopsy plasma cfDNA were compared to “ground-truth” HLA LOH results obtained from running established tumor copy number variant (CNV) calling methods on the tumor WES data. Whether running ground-truth CNV calling on the tumor DNA or running the method on the plasma cfDNA, the germline normal from each patient was used as a control for that patient's HLA LOH calls.

For the head and neck cancer cohort, ground truth HLA LOH calls were made using two CNV callers in tandem: Sequenza and Control-FREEC. First an initial pass of Control-FREEC was run and the results were passed into Sequenza; then the results from Sequenza were passed back into Control-FREEC for a second pass. The HLA LOH ground truth calls that resulted were no LOH in HLA-A, HLA-B, or HLA-C for the first patient, denoted as Patient 20; for the second patient, denoted as Patient 21, there was a positive HLA-A LOH call, and negative calls for HLA-B and HLA-C (Table 1). Hence, from the first two patients, there was one positive ground-truth call and 5 negative ground-truth calls to test our method against.

TABLE 1

| Patient and HLA gene | HLA LOH status |
|---|---|
| HN_20_A | negative |
| HN_20_B | negative |
| HN_20_C | negative |
| HN_21_A | positive |
| HN_21_B | negative |
| HN_21_C | negative |

In the table above, HN denotes head and neck cancer, the number denotes which patient, and the letter denotes which HLA gene (HLA-A, HLA-B, or HLA-C). The only HLA LOH that was observed in the ground-truth from these patients was in HLA-A from Patient 21.

Liquid Biopsy Results from the Head and Neck Cancer Cohort from NCI

The above-described method was performed on the liquid biopsy sample from each of these patients after the cfDNA was sequenced and the data were provided. The p-value from both alleles for each gene and patient were calculated, and there was only a significant p-value for Patient 21's HLA-A alleles, indicating our method was consistent with the ground-truth for all 6 of these test cases, i.e., 1/1=100% on sensitivity and 5/5=100% on specificity (Table 2).

TABLE 2

| Patient and HLA gene | Allele | p-value |
|---|---|---|
| HN_20_A | 1 | 0.439 |
| HN_20_A | 2 | 0.402 |
| HN_20_B | 1 | 0.219 |
| HN_20_B | 2 | 0.048 |
| HN_20_C | 1 | 0.710 |
| HN_20_C | 2 | 0.803 |
| HN_21_A | 1 | 0.0001897 |
| HN_21_A | 2 | 0.0001117 |
| HN_21_B | 1 | 0.371 |
| HN_21_B | 2 | 0.308 |
| HN_21_C | 1 | 0.434 |
| HN_21_C | 2 | 0.304 |

Table 2 shows P-values for each allele, HLA gene, and patient in the head and neck cancer NCI cohort. These were the first samples tested with the described embodiment of the method for HLA LOH detection via liquid biopsies. Both alleles from Patient 21's HLA-A gene showed a significant p-value, consistent with the HLA LOH positive call in the ground truth. The remaining p-values were not significant, consistent with the HLA LOH negative calls for the other test cases from both patients.

Ground-Truth from the Prostate Cancer Cohort from dbGaP

Similar to the head and neck cancer cohort from NCI, tumor copy number data were used from established tumor CNV callers. For the dbGaP cohorts (prostate cancer and breast cancer), WES data were already generated. The WES data were downloaded in the form of fastq files from dbGaP. The data available for download included tumor WES data, plasma cfDNA WES data, and germline WES data from each patient. There were 14 patients from whom these sets of three samples were sequenced and available for download.

For the prostate cancer and breast cancer dbGaP cohorts, CNV calls were already made on the tumors and were available via the publication's supplementary data. CNV calls were therefore previously generated and published. Two tumor CNV callers, ABSOLUTE and TITAN, were used. To find the HLA LOH calls from the ground-truth ABSOLUTE and TITAN tumor output, the ABSOLUTE and TITAN output files were looked at, which were excel files listing CNV events by genomic coordinate. HLA LOH results were identified within these files by looking at the CNVs that included the HLA genes' genomic coordinates according to hg38 which have the following start locations and extend approximately 3,000 bases (Table 3):

TABLE 3

| | Start location (bp) |
|---|---|
| Gene | on Chromosome 6 |
| HLA-A | 29942532 |
| HLA-B | 31353875 |
| HLA-C | 31268749 |

Table 3 shows the locations of the HLA class I genes tested in this embodiment of the method. Notice that HLA-C is located in between HLA-A and HLA-B. Since the tumor CNV callers do not return gene-specific LOH or, more generally, CNV calls, the genomic coordinates of the HLA genes were used to identify HLA LOH in the tumor samples from the WES data.

The HLA LOH results from the “ground-truth” tumor CNV callers are listed in Table 4. HLA calls were considered positive if both tumor CNV callers showed LOH for a given HLA gene, negative if both tumor CNV callers showed no LOH for a given HLA gene, and any HLA LOH calls that were inconsistent between the two tumor CNV callers were not used as ground-truth. In the following table, any genes that are missing from a given patient were not tested, either due to having homozygous alleles or having tumor HLA LOH calls that were inconsistent between the two tumor CNV callers (ABSOLUTE and TITAN). That is why even though there were 43 patients in our validation data set, there are only 107 test cases rather than 43×3=129 test cases.

TABLE 4

| Patient and HLA gene | HLA LOH status |
|---|---|
| PC_161_A | negative |
| PC_264_A | negative |
| PC_264_B | negative |
| PC_264_C | negative |
| PC_342_A | positive |
| PC_342_B | positive |
| PC_342_C | positive |
| PC_362_A | negative |
| PC_362_B | negative |
| PC_362_C | negative |
| PC_372_A | negative |
| PC_372_B | negative |
| PC_372_C | negative |
| PC_463_B | negative |
| PC_463_C | negative |
| PC_466_B | negative |
| PC_466_C | negative |
| PC_468_A | negative |
| PC_468_B | negative |
| PC_468_C | negative |
| PC_525_A | negative |
| PC_525_B | negative |
| PC_525_C | negative |
| PC_531_A | negative |
| PC_531_B | negative |
| PC_531_C | negative |
| PC_554_A | negative |
| PC_554_B | negative |
| PC_17_A | negative |
| PC_17_B | negative |
| PC_17_C | negative |
| PC_22_A | negative |
| PC_22_B | negative |
| PC_22_C | negative |

In Table 4, PC=prostate cancer, number is the patient number, and A, B, or C indicates HLA-A, HLA-B, or HLA-C, respectively. There were 3 positives in the ground-truth, all from the same patient. All other HLA LOH calls were negative. This is somewhat consistent with the prevalence of HLA LOH in prostate cancer of 9%, i.e., the expected fraction of prostate cancer patients to have HLA LOH in their tumor(s) is 1/11, and here HLA was were observed in 1 of the 14 patients. In head and neck cancer, the prevalence of HLA LOH is 28% and in breast cancer the prevalence is 16%.

Liquid Biopsy Results from the Prostate Cancer Cohort from dbGaP

The method described herein was used on the liquid biopsy samples and the results were compared to the tumor ground-truth HLA LOH calls listed above. The results are shown below (Table 5); the specificity was 31/31=100% on the HLA LOH negative calls, and the sensitivity was 3/3=100% on the HLA LOH positive calls, based on a p-value cutoff of 0.004 and using the rule that a positive call is made only if both alleles have a p-value below 0.004 and otherwise the call is negative.

TABLE 5

| Patient and HLA gene | Allele | p-value |
|---|---|---|
| PC_161_A | 1 | 0.02391 |
| PC_161_A | 2 | 0.008911 |
| PC_264_A | 1 | 0.01398 |
| PC_264_A | 2 | 0.01874 |
| PC_264_B | 1 | 0.2238 |
| PC_264_B | 2 | 0.3069 |
| PC_264_C | 1 | 0.7208 |
| PC_264_C | 2 | 0.3783 |
| PC_342_A | 1 | 9.907e−05 |
| PC_342_A | 2 | 0.0003904 |
| PC_342_B | 1 | 0.002185 |
| PC_342_B | 2 | 0.003102 |
| PC_342_C | 1 | 0.0006803 |
| PC_342_C | 2 | 0.0001716 |
| PC_362_A | 1 | 0.9652 |
| PC_362_A | 2 | 0.9764 |
| PC_362_B | 1 | 0.01806 |
| PC_362_B | 2 | 0.03097 |
| PC_362_C | 1 | 0.2704 |
| PC_362_C | 2 | 0.7555 |
| PC_372_A | 1 | 0.009548 |
| PC_372_A | 2 | 0.0139 |
| PC_372_B | 1 | 0.05807 |
| PC_372_B | 2 | 0.05807 |
| PC_372_C | 1 | 0.03491 |
| PC_372_C | 2 | 0.06373 |
| PC_463_B | 1 | 0.1525 |
| PC_463_B | 2 | 0.1094 |
| PC_463_C | 1 | 0.3655 |
| PC_463_C | 2 | 0.3745 |
| PC_466_B | 1 | 0.5874 |
| PC_466_B | 2 | 0.8094 |
| PC_466_C | 1 | 0.00527 |
| PC_466_C | 2 | 0.001496 |
| PC_468_A | 1 | 0.2381 |
| PC_468_A | 2 | 0.2411 |
| PC_468_B | 1 | 0.1088 |
| PC_468_B | 2 | 0.1167 |
| PC_468_C | 1 | 0.5633 |
| PC_468_C | 2 | 0.5293 |
| PC_525_A | 1 | 0.0463 |
| PC_525_A | 2 | 0.04193 |
| PC_525_B | 1 | 0.1651 |
| PC_525_B | 2 | 0.1335 |
| PC_525_C | 1 | 0.6093 |
| PC_525_C | 2 | 0.4577 |
| PC_531_A | 1 | 0.1131 |
| PC_531_A | 2 | 0.128 |
| PC_531_B | 1 | 0.4358 |
| PC_531_B | 2 | 0.5984 |
| PC_531_C | 1 | 0.2022 |
| PC_531_C | 2 | 0.2977 |
| PC_554_A | 1 | 0.1698 |
| PC_554_A | 2 | 0.1379 |
| PC_554_B | 1 | 0.7858 |
| PC_554_B | 2 | 0.7297 |
| PC_17_A | 1 | 0.6385 |
| PC_17_A | 2 | 0.5989 |
| PC_17_B | 1 | 0.7188 |
| PC_17_B | 2 | 0.8843 |
| PC_17_C | 1 | 0.4743 |
| PC_17_C | 2 | 0.1269 |
| PC_22_A | 1 | 0.3214 |
| PC_22_A | 2 | 0.2815 |
| PC_22_B | 1 | 0.4963 |
| PC_22_B | 2 | 0.1558 |
| PC_22_C | 1 | 0.008678 |
| PC_22_C | 2 | 0.01055 |

Table 5 shows p-values for both alleles of each HLA gene that was tested for each patient. All of the liquid biopsy HLA LOH calls from the prostate cancer dbGaP cohort were consistent with ground-truth. There were 3 positive HLA LOH calls, corresponding to all three of Patient 342's HLA class I genes (HLA-A, HLA-B, and HLA-C).

Ground-Truth from the Breast Cancer Cohort from dbGaP

Ground truth HLA LOH calls from the dbGaP breast cancer cohort were made in the same way that HLA LOH calls were made for the dbGaP prostate cancer cohort, i.e., from ABSOLUTE and TITAN calls made previously by the authors who published the data. In the breast cancer cohort there were tumor DNA, plasma cfDNA, and germline DNA WES data from 27 patients. One of the germline DNA WES samples could not be analyzed for HLA typing due to small sequencing file size. Accordingly, the data from this patient could not be utilized for validation of the HLA LOH liquid biopsy detection method. Ground truth HLA LOH was determined from each patient's tumor based on the genomic coordinates (see Table 3 above) from the ABSOLUTE and TITAN CNV calling data, as was performed for the prostate cancer dbGaP data set.

The ground truth HLA LOH calls are listed in Table 6 below. As with the prostate cancer dbGaP data set, HLA calls were considered positive if both tumor CNV callers showed LOH for a given HLA gene, negative if both tumor CNV callers showed no LOH for a given HLA gene, and any HLA LOH calls that were inconsistent between the two tumor CNV callers were not used as ground-truth. Again, in the following table, any genes that are missing from a given patient were not tested, either due to having homozygous alleles or having tumor HLA LOH calls that were inconsistent between the two tumor CNV callers (ABSOLUTE and TITAN).

TABLE 6

| Patient and HLA gene | HLA LOH status |
|---|---|
| MBC_191_A | negative |
| MBC_191_B | negative |
| MBC_191_C | negative |
| MBC_217_B | negative |
| MBC_284_A | negative |
| MBC_284_B | negative |
| MBC_284_C | negative |
| MBC_287_A | negative |
| MBC_287_B | negative |
| MBC_287_C | negative |
| MBC_288_A | negative |
| MBC_288_B | negative |
| MBC_288_C | negative |
| MBC_291_A | negative |
| MBC_291_B | negative |
| MBC_291_C | negative |
| MBC_292_A | negative |
| MBC_292_B | negative |
| MBC_292_C | negative |
| MBC_295_A | negative |
| MBC_299_A | negative |
| MBC_299_B | negative |
| MBC_301_A | negative |
| MBC_301_B | negative |
| MBC_301_C | negative |
| MBC_303_B | negative |
| MBC_303_C | negative |
| MBC_307_A | negative |
| MBC_307_B | negative |
| MBC_307_C | negative |
| MBC_313_B | negative |
| MBC_313_C | negative |
| MBC_315_A | positive |
| MBC_315_B | positive |
| MBC_315_C | positive |
| MBC_317_A | positive |
| MBC_317_B | positive |
| MBC_317_C | positive |
| MBC_318_A | positive |
| MBC_318_B | positive |
| MBC_318_C | positive |
| MBC_320_A | negative |
| MBC_320_B | negative |
| MBC_325_A | negative |
| MBC_325_B | negative |
| MBC_325_C | negative |
| MBC_330_B | negative |
| MBC_330_C | negative |
| MBC_331_A | negative |
| MBC_331_B | negative |
| MBC_331_C | negative |
| MBC_333_A | negative |
| MBC_333_B | negative |
| MBC_333_C | negative |
| MBC_335_A | negative |
| MBC_335_B | negative |
| MBC_336_A | negative |
| MBC_336_B | negative |
| MBC_336_C | negative |
| MBC_339_A | negative |
| MBC_339_B | positive |
| MBC_339_C | positive |
| MBC_349_A | negative |
| MBC_349_B | negative |
| MBC_349_C | negative |
| MBC_8_A | negative |
| MBC_8_C | negative |

In Table 6, MBC=metastatic breast cancer, number is the patient number, and A, B, or C indicates HLA-A, HLA-B, or HLA-C, respectively. Out of 26 patients, 4 patients had at least one HLA LOH call (4/26=0.154), which is somewhat consistent with the prevalence of HLA LOH in breast cancer, which is 16%.

Liquid Biopsy Results from the Breast Cancer Cohort from dbGaP

The method described above was performed on the liquid biopsy samples and the results were compared the results to the tumor ground-truth HLA LOH calls listed above in Table 6. The results are shown below (Table 7). In the described embodiment, the criterion for calling HLA LOH positive from a given patient's liquid biopsy sample is a p-value less than 0.004 for both alleles of a given HLA gene (HLA-A, HLA-B, or HLA-C).

TABLE 7

| Patient and HLA gene | Allele | p-value |
|---|---|---|
| MBC_191_A | 1 | 0.9538 |
| MBC_191_A | 2 | 0.9472 |
| MBC_191_B | 1 | 0.4808 |
| MBC_191_B | 2 | 0.9566 |
| MBC_191_C | 1 | 0.9227 |
| MBC_191_C | 2 | 0.9593 |
| MBC_217_B | 1 | 0.08949 |
| MBC_217_B | 2 | 0.09165 |
| MBC_284_A | 1 | 0.06107 |
| MBC_284_A | 2 | 0.03736 |
| MBC_284_B | 1 | 0.4258 |
| MBC_284_B | 2 | 0.3946 |
| MBC_284_C | 1 | 0.00178 |
| MBC_284_C | 2 | 0.004221 |
| MBC_287_A | 1 | 0.05902 |
| MBC_287_A | 2 | 0.05138 |
| MBC_287_B | 1 | 0.01254 |
| MBC_287_B | 2 | 0.006317 |
| MBC_287_C | 1 | 0.9839 |
| MBC_287_C | 2 | 0.9839 |
| MBC_288_A | 1 | 0.4147 |
| MBC_288_A | 2 | 0.3282 |
| MBC_288_B | 1 | 0.07035 |
| MBC_288_B | 2 | 0.08706 |
| MBC_288_C | 1 | 0.1899 |
| MBC_288_C | 2 | 0.195 |
| MBC_{—}291_{—}A | 1 | 1.206e−05 |
| MBC_{—}291_{—}A | 2 | 1.539e−05 |
| MBC_291_B | 1 | 0.08939 |
| MBC_291_B | 2 | 0.0478 |
| MBC_291_C | 1 | 0.01282 |
| MBC_291_C | 2 | 0.01091 |
| MBC_292_A | 1 | 0.6039 |
| MBC_292_A | 2 | 0.7602 |
| MBC_292_B | 1 | 0.001864 |
| MBC_292_B | 2 | 0.06088 |
| MBC_292_C | 1 | 0.06005 |
| MBC_292_C | 2 | 0.1079 |
| MBC_295_A | 1 | 0.004257 |
| MBC_295_A | 2 | 0.001547 |
| MBC_299_A | 1 | 0.1836 |
| MBC_299_A | 2 | 0.1405 |
| MBC_299_B | 1 | 0.7683 |
| MBC_299_B | 2 | 0.4102 |
| MBC_301_A | 1 | 0.2604 |
| MBC_301_A | 2 | 0.4073 |
| MBC_301_B | 1 | 0.00433 |
| MBC_301_B | 2 | 0.001358 |
| MBC_{—}301_{—}C | 1 | 0.001458 |
| MBC_{—}301_{—}C | 2 | 0.001033 |
| MBC_303_B | 1 | 0.9284 |
| MBC_303_B | 2 | 0.8855 |
| MBC_303_C | 1 | 0.5953 |
| MBC_303_C | 2 | 0.5195 |
| MBC_{—}307_{—}A | 1 | 5.541e−07 |
| MBC_{—}307_{—}A | 2 | 5.075e−07 |
| MBC_{—}307_{—}B | 1 | 0.0003496 |
| MBC_{—}307_{—}B | 2 | 0.001062 |
| MBC_307_C | 1 | 0.01222 |
| MBC_307_C | 2 | 0.007522 |
| MBC_313_B | 1 | 0.9417 |
| MBC_313_B | 2 | 0.7653 |
| MBC_313_C | 1 | 0.2114 |
| MBC_313_C | 2 | 0.1818 |
| MBC_{—}315_{—}A | 1 | 0.007653 |
| MBC_{—}315_{—}A | 2 | 0.01674 |
| MBC_{—}315_{—}B | 1 | 0.009307 |
| MBC_{—}315_{—}B | 2 | 0.9445 |
| MBC_{—}315_{—}C | 1 | 0.973 |
| MBC_{—}315_{—}C | 2 | 0.8111 |
| MBC_317_A | 1 | 0.0002037 |
| MBC_317_A | 2 | 0.0002885 |
| MBC_317_B | 1 | 1.044e−05 |
| MBC_317_B | 2 | 1.144e−05 |
| MBC_317_C | 1 | 0.0005355 |
| MBC_317_C | 2 | 0.001397 |
| MBC_318_A | 1 | 0.0001497 |
| MBC_318_A | 2 | 3.277e−05 |
| MBC_318_B | 1 | 0.001589 |
| MBC_318_B | 2 | 0.001635 |
| MBC_318_C | 1 | 0.000212 |
| MBC_318_C | 2 | 0.0005172 |
| MBC_{—}320_{—}A | 1 | 0.002405 |
| MBC_{—}320_{—}A | 2 | 0.001636 |
| MBC_320_B | 1 | 0.1882 |
| MBC_320_B | 2 | 0.2074 |
| MBC_325_A | 1 | 0.455 |
| MBC_325_A | 2 | 0.3486 |
| MBC_325_B | 1 | 0.442 |
| MBC_325_B | 2 | 0.1315 |
| MBC_325_C | 1 | 0.4161 |
| MBC_325_C | 2 | 0.1545 |
| MBC_330_B | 1 | 0.1976 |
| MBC_330_B | 2 | 0.2625 |
| MBC_330_C | 1 | 0.7387 |
| MBC_330_C | 2 | 0.4526 |
| MBC_331_A | 1 | 0.01423 |
| MBC_331_A | 2 | 0.008403 |
| MBC_{—}331_{—}B | 1 | 0.0001943 |
| MBC_{—}331_{—}B | 2 | 2.799e−05 |
| MBC_331_C | 1 | 0.2742 |
| MBC_331_C | 2 | 0.1626 |
| MBC_333_A | 1 | 0.08061 |
| MBC_333_A | 2 | 0.08012 |
| MBC_333_B | 1 | 0.3333 |
| MBC_333_B | 2 | 0.2032 |
| MBC_333_C | 1 | 0.04438 |
| MBC_333_C | 2 | 0.06763 |
| MBC_{—}335_{—}A | 1 | 2.801e−07 |
| MBC_{—}335_{—}A | 2 | 2.785e−07 |
| MBC_{—}335_{—}B | 1 | 4.959e−08 |
| MBC_{—}335_{—}B | 2 | 2.033e−07 |
| MBC_336_A | 1 | 0.06107 |
| MBC_336_A | 2 | 0.06635 |
| MBC_336_B | 1 | 0.1211 |
| MBC_336_B | 2 | 0.1454 |
| MBC_336_C | 1 | 0.07468 |
| MBC_336_C | 2 | 0.08914 |
| MBC_339_A | 1 | 0.009401 |
| MBC_339_A | 2 | 0.01013 |
| MBC_{—}339_{—}B | 1 | 0.1625 |
| MBC_{—}339_{—}B | 2 | 0.6764 |
| MBC_{—}339_{—}C | 1 | 0.9206 |
| MBC_{—}339_{—}C | 2 | 0.7666 |
| MBC_349_A | 1 | 0.1312 |
| MBC_349_A | 2 | 0.1415 |
| MBC_349_B | 1 | 0.5737 |
| MBC_349_B | 2 | 0.1324 |
| MBC_349_C | 1 | 0.9427 |
| MBC_349_C | 2 | 0.9377 |
| MBC_8_A | 1 | 0.05068 |
| MBC_8_A | 2 | 0.05068 |
| MBC_8_C | 1 | 0.4735 |
| MBC_8_C | 2 | 0.6355 |

Table 7 shows p-values for both alleles from each gene that was tested for each patient in the dbGaP breast cancer cohort. Bold marks false positives and italics marks false negatives. There were 5 test cases with false negatives and 8 test cases with false positives in this cohort, whereas the first two cohorts did not have any false positives or false negatives.

The false positives and false negatives in this cohort may have been caused by sample quality issues; it was observed that at least one germline sample from this cohort had low read count and file size for the fastq files, and it is possible that some of the plasma samples also had quality issues in the sample preparation and/or sequencing. Another possibility is that the described embodiment of the method must be improved, potentially leading to improved embodiments of the method for HLA LOH detection from liquid biopsies. In the dbGaP breast cancer cohort, the sensitivity from the described embodiment was 6/11 and the specificity was 48/56. Overall, considering all three cohorts on which the described embodiment was tested, the sensitivity was 10/15 and the specificity was 84/92. It is possible there were sample quality issues in this third cohort.

### Discussion

It is clear that the described embodiment of the method for detection of HLA LOH from liquid biopsies has the ability to detect true positives and true negatives. That being said, it is possible that there were issues with the sample quality or library prep or sequencing quality in connection with the dbGaP breast cancer cohort. Further testing will be required to determine the cause(s) of the false positives and false negatives in that cohort.

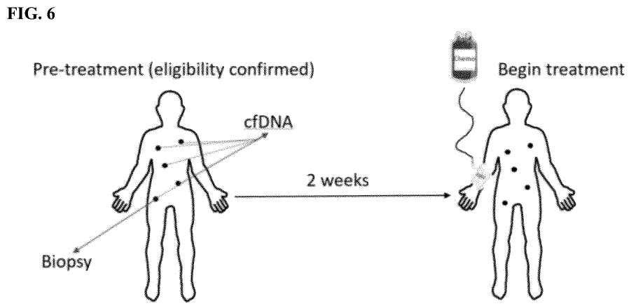

The above-described method may be used detect HLA LOH from liquid biopsies as a biomarker that can be utilized to predict response or resistance to TCR-based therapies. One embodiment of that usage as a biomarker in a diagnostic assay is to be able to make a treatment decision quickly, i.e., within two weeks of a patient's enrollment on a clinical study for TCR-T cell therapy (FIG. 6). This will enable fast treatment of a patient if they do not have HLA LOH, to give the patient the best chance of survival, and will lead to avoidance of the potentially toxic chemotherapy conditioning regimen that is part of the protocol for all TCR-T cell therapies currently.

FIG. 6 shows one potential usage of the method described herein in an assay that may be classified as a clinical trial assay (CTA) that helps determine enrollment on clinical studies for TCR-T cell therapies. A two-week turnaround time from liquid biopsy sample collection to HLA LOH results might be the goal, as this is the current usual time frame from enrollment to treatment due to the manufacturing time for the cell therapy. The treatment with TCR-T cell therapy begins with a chemotherapy conditioning regimen that aids in allowing the engineered infused T cells to target and destroy cancer cells (assuming the cancer does not have HLA LOH). Simultaneously, in the trial phase of the diagnostic, a solid tissue biopsy can also be collected to ensure consistency between the liquid biopsy HLA LOH results and the solid tumor HLA LOH results. The patient can then be examined longitudinally (over the course of a year or two) to determine if the treatment was effective, and this information can also be utilized for determining whether there is a true correlation between HLA LOH and resistance for this type of therapy.

It has been shown previously that immune checkpoint inhibition (ICI) therapies may be more effective if HLA LOH is utilized as a biomarker in addition to tumor mutation burden-high (TMB-H) status. In that context, the method described herein could be utilized as an additional companion diagnostic for ICI therapy. The method is also applicable to the other therapeutic modalities listed above, including T-cell engagers, cancer vaccines, cytokine therapy, and TIL therapy because all of these require HLA intactness in order for the therapeutic to be effective.

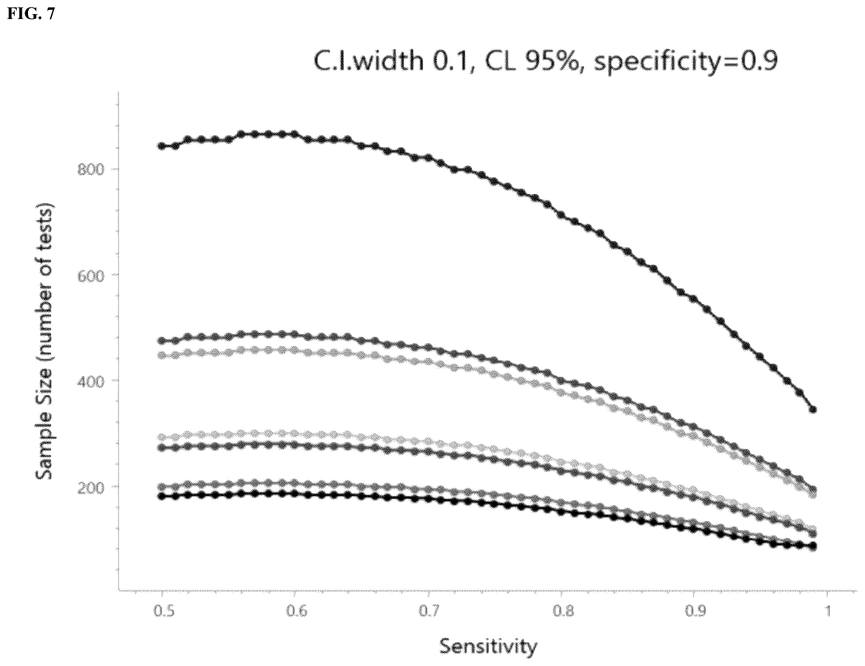

Statistical analyses were performed to determine how many test cases will be required to ensure the tests sensitivity and specificity to a given level of confidence. For example, if the test has an inherent sensitivity of 95% and specificity of 90%, then approximately 300 test cases will lead us to calculate the sensitivity and specificity at approximately 95% and 90%, respectively, with a confidence level of 95% (FIG. 7).

FIG. 7 shows a sample size calculation using a one-side (lower) 95% exact Clopper-Pearson confidence interval with confidence interval width of 0.1. The pan-cancer prevalence of HLA LOH is 0.17 (orange curve), meaning that for samples randomly selected from patients with any type of cancer, the required sample size is ˜300 if the true sensitivity and specificity of the HLA LOH detection test are 95% and 90%, respectively. Other curves represent sample sizes if the test were performed within only one cancer type, with the prevalences shown. The HLA LOH prevalence in prostate cancer is 0.09; prevalence of 0.16 corresponds to breast cancer; 0.26 is the prevalence in pancreatic carcinoma; 0.28 is the prevalence in head and neck cancer; 0.38 is the prevalence in pancreatic islet cell neuroendocrine cancer; 0.42 is the prevalence in thymic cancer.

### CONCLUSION

The results of performing the method for HLA LOH detection from liquid biopsies described above demonstrate that it works. Surprisingly, the method worked properly to detect HLA LOH in dozens of samples. It was previously thought impossible to detect HLA LOH in liquid biopsies, because of low tumor fraction in cell-free DNA as well as it being thought that detecting a negative would be difficult. That is, it would be difficult to detect a loss of a given genomic feature (in this case, HLA genes), rather than an amplification or presence of a given genomic feature, which most liquid biopsy assays today detect—in the form of mutations and gene fusions. The method described herein represents one of the first, if not the first, methods for detecting a genomic copy loss biomarker for use in treatment decision-making for cancer.

What is claimed is:

1. A method of detecting human leukocyte antigen (HLA) gene loss of heterozygosity (LOH) in a subject, comprising
(a) providing biopsy HLA sequence data obtained from cell-free DNA (cfDNA) of a liquid biopsy sample from the subject, wherein the biopsy HLA sequence data comprises sequences of one or more HLA genes, whole genome sequence (WGS) data, or whole exome sequence (WES) data;
(b) providing germline HLA sequence data obtained from DNA of a germline cell sample of the subject, wherein the germline HLA sequence data comprises sequences of one or more HLA genes, WGS data, or WES data, and wherein each of one or more HLA genes from the germline cell DNA is genotyped to two-digit, four-digit, six-digit, or eight-digit resolution;
(c) aligning coding sequences of both alleles of each HLA gene from the germline cell DNA to a reference sequence for each HLA gene to generate an alignment, and aligning cfDNA coding sequences to a reference genome so that reads at heterozygous bases can be counted;
(d) from the alignment, identifying heterozygous bases of each HLA gene, wherein each heterozygous base is identified by coding sequence position and genome coordinate;
(e) for each heterozygous base of each HLA gene, counting allelic reads in the biopsy HLA sequence data and counting allelic reads in the germline HLA sequence data;
(f) calculating a weight for each heterozygous base by:
(i) separately for allelic reads in the biopsy HLA sequence data and the germline HLA sequence data, for each heterozygous base of each HLA gene, dividing the read count of each heterozygous base by the sum of read counts on all heterozygous bases; and
(ii) calculating the average at each heterozygous base between the biopsy HLA sequence data and the germline HLA sequence data;
(g) comparing the allelic read count from the biopsy HLA sequence data to the allelic read count from the germline HLA sequence data, by performing a Student's t-test with paired observation for each heterozygous base, in which each weight is multiplied by an observation comprising the read count at the heterozygous base in the biopsy HLA sequence data and the read count at the same heterozygous base in the germline DNA sequence data, to generate a p-value for each HLA gene;
wherein a significant p-value, which optionally is 0.004, is indicative of LOH for a HLA gene in the liquid biopsy.

2. The method of claim 1, wherein the germline cell sample comprises peripheral blood monocytes.

3. The method of claim 1 or 2, wherein the subject has a cancer, and wherein the cfDNA comprises cancer cell DNA.

4. A method of predicting a subject's response to a cancer treatment that targets an antigen bound to a restriction element encoded by a HLA gene, comprising detecting LOH for the HLA gene according to the method of claim 1, wherein the presence of LOH for the HLA gene is indicative that the subject will have a poor response to the cancer treatment, and wherein the absence of LOH for the HLA gene is indicative that the subject will respond or is more likely to respond to the cancer treatment.

5. The method of claim 4 wherein the cancer treatment is a T cell receptor (TCR)-based therapy.

6. The method of claim 5, wherein the TCR-based therapy is selected from the group consisting of an immune checkpoint blockade, a T cell engager, a TCR-T cell therapy, tumor infiltrating lymphocytes, a cancer vaccine, and a cytokine therapy.

7. The method of claim 6, wherein the T cell engager comprises immune mobilizing TCRs against cancer.

8. A method of treating a cancer in a subject in need thereof, comprising administering to the subject a cancer treatment that targets an antigen bound to a restriction element encoded by a HLA gene, wherein the subject has been predicted to respond or be more likely to respond to the cancer treatment according to the method of claim 4.

9. The method of claim 8, wherein the cancer treatment comprises a TCR-based therapy.

10. The method of claim 9, wherein the TCR-based therapy is selected from the group consisting of an immune checkpoint blockade, a T cell engager, a TCR-T cell therapy, tumor infiltrating lymphocytes, a cancer vaccine, and a cytokine therapy.

11. A system for detecting human leukocyte antigen (HLA) gene loss of heterozygosity (LOH) in a subject, comprising:
a computing system including a processor in communication with a memory, the memory including instructions, which, when executed, cause the processor to:
(a) provide biopsy HLA sequence data obtained from cfDNA of a liquid biopsy sample from the subject, wherein the biopsy HLA sequence data comprises sequences of one or more HLA genes, whole genome sequence (WGS) data, or whole exome sequence (WES) data;
(b) provide germline HLA sequence data obtained from DNA of a germline cell sample of the subject, wherein the germline HLA sequence data comprises sequences of one or more HLA genes, WGS data, or WES data, and wherein each of one or more HLA genes from the germline cell DNA is genotyped to two-digit, four-digit, six-digit, or eight-digit resolution, or any resolution high enough to identify heterozygous bases;
(c) align coding sequences of both alleles of each HLA gene from the germline cell DNA to a reference sequence for each HLA gene to generate an alignment; and align cfDNA coding sequences to a reference genome so that reads at heterozygous bases can be counted;
(d) from the alignment, identify heterozygous bases of each HLA gene, wherein each heterozygous base is identified by coding sequence position and genome coordinate;
(e) for each heterozygous base of each HLA gene, count allelic reads in the biopsy HLA sequence data and counting allelic reads in the germline sequence data;
(f) calculate a weight for each heterozygous base by:
(i) separately for allelic reads in the biopsy HLA sequence data and the germline HLA sequence data, for each heterozygous base of each HLA gene, dividing the read count of each heterozygous base by the sum of read counts on all heterozygous bases; and
(ii) calculating the average at each heterozygous base between the biopsy HLA sequence data and the germline HLA sequence data;
(g) compare the allelic read count from the biopsy HLA sequence data to the allelic read count from the germline HLA sequence data, by performing a Student's t-test with paired observation for each heterozygous base, in which each weight is multiplied by an observation comprising the read count at the heterozygous base in the biopsy HLA sequence data and the read count at the same heterozygous base in the germline DNA sequence data, to generate a p-value for each HLA gene;
wherein a significant p-value, which is optionally is 0.004, is indicative of LOH for a HLA gene in the liquid biopsy.

12. The system of claim 11, wherein the germline cell sample comprises peripheral blood monocytes.

13. The system of claim 11 or 12, wherein the subject has a cancer, and wherein the cfDNA comprises cancer cell DNA.

14. A system for predicting a subject's response to a cancer treatment that targets an antigen bound to a restriction element encoded by a HLA gene, comprising:
a computing system including a processor in communication with a memory, the memory including instructions, which, when executed, cause the processor to:
detect LOH for the HLA gene according to claim 1, wherein the presence of LOH for the HLA gene is indicative that the subject will have a poor response to the cancer treatment, and wherein the absence of LOH for the HLA gene detected by the processor is indicative that the subject will respond or is more likely to respond to the cancer treatment.

15. The system of claim 14, wherein the cancer treatment is a TCR-based immunotherapy.

16. A system of treating a cancer in a subject in need thereof, comprising:
a computing system including a processor in communication with a memory, the memory including instructions, which, when executed, cause the processor to:
instruct that the subject be administered a cancer treatment that targets an antigen bound to a restriction element encoded by a HLA gene, wherein the subject has been predicted by the processor to respond to the cancer treatment according to the system of claim 14.

17. The system of claim 16, wherein the cancer treatment is a TCR-based therapy.

18. The system of claim 17, wherein the TCR-based therapy is selected from the group consisting of an immune checkpoint blockade, a T cell engager, a TCR-T cell therapy, tumor infiltrating lymphocytes, a cancer vaccine, and a cytokine therapy.
